(19) United States
(12) Patent Application Publication
(10) Pub. No.: US 2013/0204015 A1
(43) Pub. Date: Aug. 8, 2013

(54) PROCESS FOR PREPARING A LACTONE

(71) Applicants: Roland Jacquot, Francheville (FR); Philippe Marion, Vernaison (FR)

(72) Inventors: Roland Jacquot, Francheville (FR); Philippe Marion, Vernaison (FR)

(73) Assignee: RHODIA OPERATIONS, Aubervilliers (FR)

(21) Appl. No.: 13/639,963

(22) PCT Filed: Apr. 5, 2011
(86) PCT No.: PCT/EP11/55289

§ 371 Date: Jan. 18, 2013

(30) Foreign Application Priority Data
Apr. 7, 2010 (FR) 10/01431

(51) Int. Cl. C07D 307/33; C07D 313/04

(52) U.S. Cl. CPC C07D 307/33; C07D 313/04
USPC 549/266; 502/325; 549/326

(57) ABSTRACT

A method for preparing a lactone is described. Also described, is the preparation of butyrolactone, valerolactone and caprolactone. The method for preparing a lactone can include a reduction of a dicarboxylic acid using hydrogen, in a gaseous phase and in the presence of an effective amount of a catalyst including an active ruthenium-tin phase including at least one Ru₂Sn₃ alloy and an Ru₃Sn₇ alloy.

The present invention relates to a process for preparing a lactone.

The invention is directed in particular toward the preparation of butyrolactone, valerolactone and caprolactone.

In the present text, the term “lactone” denotes a compound which is characterized by the presence of an ester function in a ring.

It is therefore an oxygenated heterocycle comprising a carbonyl function in the position alpha to the oxygen atom.

Lactones are compounds that find many applications in industry, especially as intermediate products for the preparation of molecules in the pharmaceutical or agrichemical fields.

Lactones may also find application as solvents or may be used in the polymer field, as monomers.

Several preparation processes are described in the literature.

One route of access to lactones consists in performing an intramolecular esterification of a difunctional compound bearing a carboxylic function and an alcohol function.

Thus, U.S. Pat. No. 6,838,577 describes the preparation of lactones comprising 4 or 5 atoms by heating the corresponding hydroxy acids, resulting in the loss of a water molecule and spontaneous cyclization (comparative example) or by heating in the presence of a catalyst such as silica or alumina, and mixtures thereof.

Certain lactones, especially γ-butyrolactone, may be prepared according to GB 583 344 from the corresponding diol by gas-phase dehydrogenation in the presence of a copper or silver catalyst.

Finally, many lactones may be prepared according to the Baeyer-Villiger reaction, by reacting a cyclic ketone with a peroxide or an organic peracid obtained from a carboxylic acid, generally acetic acid and hydrogen peroxide. In particular, DE 197 45 442 discloses the preparation of δ-valerolactone by reacting cyclopentanone and hydrogen peroxide, in the presence of a catalyst which may be a cation-exchange acidic resin (Amberlyst 15) or zeolites (H-ZSM-5, H-mordenite, USY).

Relative to the processes described in the prior art, the object of the present invention is to provide a novel lactone preparation process that involves an entirely different substrate.

A process, which constitutes the subject of the present invention, has now been found for preparing a lactone, characterized in that it comprises the reduction of a dicarboxylic acid using hydrogen, in the gas phase and in the presence of an effective amount of a catalyst comprising a ruthenium-tin active phase composed at least of an alloy Ru₂Sn₃ and of an alloy Ru₃Sn₇.

Another subject of the present invention is the cyclizing hydrogenation catalyst involved in the process of the invention.

In accordance with the process of the invention, use is made of a dicarboxylic acid corresponding more particularly to formula (I) below:

HOOC—R—COOH (I)

in said formula (I), R represents a substituted or unsubstituted divalent group, comprising a linear sequence of atoms in a sufficient number to form the desired lactone.

The term “sequence of atoms” means the atoms included in the ring, the substituents being excluded.

Generally, the group R comprises a linear sequence of 2 to 8 atoms, preferably from 2 to 6 atoms and even more preferentially from 2 to 4 atoms. It is usually a sequence of carbon atoms, but the invention does not exclude the possibility of the hydrocarbon chain being interrupted with a heteroatom, especially nitrogen, oxygen or sulfur.

As mentioned previously, the divalent group R may be substituted, i.e. the hydrogen atoms of the hydrocarbon chain may be replaced with an organic group or function. Any substituent may be present, provided that it does not interfere in the cyclization reaction. In particular, the hydrocarbon chain may bear a substituent, for instance a hydroxyl group or a halogen atom, preferably fluorine, chlorine or bromine, or may bear side chains or branches that may consist, preferably, of alkyl groups generally containing from 1 to 4 carbon atoms. The branches are usually located on one or both of the carbon atoms in the position α or β to the carboxylic groups.

Overall, the group R has a total carbon condensation that may vary widely from 2 carbon atoms up to a number that may be as high as 15 carbon atoms when substituents are present and said group comprises a linear sequence of 2 to 8 atoms which is then included in the ring obtained.

In formula (I), R preferably represents a saturated or unsaturated, linear or branched divalent aliphatic group.

More precisely, R represents a saturated linear or branched aliphatic group preferably containing from 2 to 15 carbon atoms or an unsaturated linear or branched group comprising one or more unsaturations on the chain, generally 1 or 2 unsaturations which may preferably be simple or conjugated double bonds.

Dicarboxylic acids of general formula (I) in which the aliphatic group R is a linear or branched alkylene group containing from 2 to 12 carbon atoms comprising a linear sequence of 2 to 8 carbon atoms between the two COOH groups are most particularly suitable for performing the process of the invention.

The preferred group R comprises a linear sequence of 2 to 4 carbon atoms between the two COOH groups.

It is also possible to make use in the process of the invention of a dicarboxylic acid of formula (I) in which R represents a saturated or unsaturated, linear or branched aliphatic group in which two vicinal carbon atoms may form a ring.

The term “ring” means a saturated, unsaturated or aromatic carbocyclic or heterocyclic ring.

Examples of rings that may be envisioned include cycloaliphatic, aromatic and heterocyclic rings, especially cycloalkyl rings comprising 6 carbon atoms in the ring, or benzenic rings, these rings themselves possibly bearing one or more substituents provided that they do not interfere with the cyclization reaction.

Examples of such groups R that may be mentioned, inter alia, include the following groups:

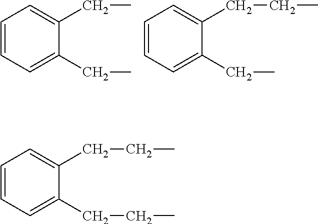

As carboxylic acids of formula (I) that are suitable for the present invention, use is made more particularly of the following dicarboxylic acids:
succinic acid,
2-ethylsuccinic acid,
glutaric acid,
2-methylglutaric acid,
2-ethylglutaric acid
adipic acid
2-methyladipic acid
3-methyladipic acid,
4-methyladipic acid,
5-methyladipic acid,
2,2-dimethyladipic acid,
3,3-dimethyladipic acid,
2,2,5-trimethyladipic acid,
2,5-dimethyladipic acid,
pimelic (heptanedioic) acid,
2-methylpimelic acid,
2,2-dimethylpimelic acid,
3,3-dimethylpimelic acid,
2,5-dimethylpimelic acid,
2,2,5-trimethylpimelic acid,
azelaic acid,
sebacic acid,
1,2-phenylenediacetic acid.

Among the abovementioned acids, succinic acid, glutaric acid and malic acid are preferred acids.

In accordance with the process of the invention, the reaction for cyclization of the dicarboxylic acid is performed in the presence of the catalyst of the invention, which is a cyclizing hydrogenation catalyst.

The active phase of the catalyst of the invention comprises ruthenium-tin alloy phases.

The ruthenium and tin are advantageously in the form of an Ru₂Sn₃ alloy mixed with the Ru₃Sn₇ alloy.

It is desirable for at least 90%, advantageously at least 95% and preferably 98% by mass of the ruthenium to be in an alloy form.

Advantageously, the active phase comprising ruthenium and tin has an Sn/Ru atomic ratio at least equal to 3/2 and preferably to 9/5.

Moreover, it is preferable for the Sn/Ru atomic ratio to be less than 7/3, advantageously 6.5/3 and even more preferentially 2/1.

Given the Sn/Ru atomic ratios in the active phase, it follows that the active phase consists predominantly of the Ru₂Sn₃ alloy phase.

The term “predominantly” means that the active phase comprises at least 75% by mass of the Ru₂Sn₃ alloy, the composition of the other fraction of the active phase depending on the Sn/Ru atomic ratio.

The Sn/Ru atomic ratio equal to 1.5 corresponds theoretically to an active phase of pure Ru₂Sn₃.

When, in the active phase, the Sn/Ru atomic ratio is greater than 1.5, the Ru₂Sn₃ alloy phase is accompanied by the Ru₃Sn₇ alloy phase.

In this case, it is advantageous for the Ru₂Sn₃ phase to represent at least 75% by mass and preferably at least 90% by mass of the two alloy phases Ru₂Sn₃ and Ru₃Sn₇.

When, in the active phase, the Sn/Ru atomic ratio decreases and becomes lower than 1.5, the Ru₂Sn₃ and Ru₃Sn₇ alloy phases are accompanied by a metallic ruthenium phase.

In the catalyst of the invention, it is advantageous for the metallic ruthenium phase to represent less than 10% by mass of the ruthenium-tin active phase.

The invention also includes the case where the active phase simultaneously comprises the Ru₂Sn₃ and Ru₃Sn₇ alloy phases and metallic ruthenium.

The invention does not exclude the case of the presence of other compounds (for instance ruthenium oxide) in minor amounts representing less than 10% by mass and preferably less than 5% of the active phase.

Although it is not excluded to use a bulk catalyst, it is preferable to deposit this active phase onto a support.

Several imperatives preside over the choice of the support.

The support must be chosen so as to maximize the resistance to industrial conditions, and in particular the resistance to mechanical abrasion, in particular the resistance to attrition.

The support must be chosen so as to avoid substantial losses of pressure, while at the same time enabling good contact between the gases and the catalyst.

The support must be inert with respect to the reaction mixture.

The support must be chosen from compounds or compositions that induce few or no side reactions.

The support may be in any form, for example powder, beads, granules, extrudates, etc.

As supports that are suitable for use in the catalyst of the invention, mention may be made, inter alia, of metal oxides.

Thus, the support may be chosen especially from metal oxides, such as aluminum, silicon, titanium and/or zirconium oxides, or mixtures thereof.

Mixed oxides are also suitable for use, and more particularly those containing at least ¼, advantageously ⅓ and preferably ⅖ by mass of aluminum expressed as Al₂O₃.

It is desirable for the support advantageously to have a silicon content which, expressed as SiO₂, is not more than ⅔ and advantageously not more than ¼ of the total weight.

It should be noted that the specific surface area, BET, of the support is advantageously chosen between 5 and 100 m²/g and preferably between 10 and 50 m²/g.

For the definition of the specific surface area, reference is made to the Brunauer-Emmett-Teller method described in the Journal of the American Chemical Society, 60, 309 (1938).

If the catalytic phase is deposited onto a support, the ruthenium content of the catalyst is advantageously chosen between 1% and 8% by mass and even more preferentially between 2% and 3% by mass.

The catalyst included in the process of the invention is a cyclizing hydrogenation catalyst comprising an active phase which has characteristics that are intrinsic thereto:
said ruthenium-tin active phase is composed at least of an Ru₂Sn₃ alloy and of an Ru₃Sn₇ alloy,
the Ru₂Sn₃ alloy phase represents at least 75% by mass of the ruthenium-tin active phase,
at least 90% by mass of the ruthenium is in an Ru₂Sn₃ and Ru₃Sn₇ alloy form.

In the preferred catalyst, the Ru₂Sn₃ alloy phase represents at least 90% by mass of the two alloy phases Ru₂Sn₃ and Ru₃Sn₇.

In the preferred catalyst, ruthenium is present in an alloy form to at least 90%, preferably to at least 95% and even more preferentially to at least 98%.

One of the modes of preparation of said ruthenium-tin catalyst consists in reducing a ruthenium complex having an electrovalency of −4 and a coordination number of 6, the coordinates being either a halogen atom or a tin halide anion.

According to one preferred mode of the process of the invention, reduction is performed on a complex more particularly corresponding to formula (A) below:

[Ru(SnX₃)₆₋ₙXₙ]⁴⁻ (A)

in said formula (A), X represents a halogen atom, preferably a chlorine or bromine atom, and n is a number equal to 1 or 2 and preferably equal to 2.

The following complexes are preferentially involved in the process of the invention:

[Ru(SnCl₃)₅Cl]⁴⁻

[Ru(SnCl₃)₄Cl₂]⁴⁻

According to one preferred embodiment of the invention, the preparation of the complex(es) is performed by reacting a ruthenium halide and a tin halide, in the presence of an acid.

To this end, the starting reagent used is a ruthenium III halide, preferably a ruthenium III chloride. It is also possible to start with a ruthenium IV salt, but there is no additional advantage and, what is more, it is more expensive.

Use is thus preferentially made of a ruthenium III halide, which may be, without preference, in anhydrous or hydrated form.

It is desirable for said compound not to contain an excessive amount of impurities. Advantageously, use is made of a compound free of heavy metals and having a ruthenium chemical purity of 99% relative to the other metals.

It is possible without drawback to use the commercial form of ruthenium chloride, RuCl₃.xH₂O, comprising approximately 42% to 43% by mass of ruthenium.

As regards the tin salt, use is made of a tin halide in which the tin has an oxidation state less than that of the ruthenium.

A tin II halide, preferably a tin II chloride, is used.

The salt may also be used in anhydrous or hydrated form. Preferentially, the commercial tin salt of formula SnCl₂.2H₂O is also used.

Usually, the halides of said metals are used in aqueous solution form. The concentration of these solutions is such that a homogeneous solution that can be impregnated onto a support is obtained.

As regards the amounts of the abovementioned metal halides employed, they are determined such that the ratio between the number of moles of tin halide and the number of moles of ruthenium halide ranges between 1 and 5 and preferably between 2 and 4.

When the ratio between the number of moles of tin halide and the number of moles of ruthenium halide is between 4 and 5, the active phase of the catalyst obtained comprises the alloy phase Ru₂Sn₃ which is accompanied by an alloy phase Ru₂Sn₇.

When the ratio between the number of moles of tin halide and the number of moles of ruthenium halide becomes less than or equal to 4, a ruthenium metallic phase appears.

There is coexistence of the three phases, the alloy phases Ru₂Sn₃ and Ru₂Sn₇ and metallic ruthenium.

The catalyst advantageously used in the process of the invention results from the use of tin and ruthenium halides such that their mole ratio is between 2 and 4.

The preparation of the complex by reaction of the ruthenium and tin halides is performed in the presence of an acid whose function is to dissolve the tin halide and to keep the formed complex soluble.

Use may be made of any strong acid, preferably a mineral acid, but it is preferred to use the hydracid whose halide is identical to the halide included in the ruthenium and tin salts.

Thus, hydrochloric acid is generally the preferred acid.

The amount of acid used is preferably at least 1 mol of acid per mole of ruthenium halide and more particularly between 1 and 5 mol of acid per mole of ruthenium halide. The upper limit is not critical and may be exceeded without drawback. The preferred amount of acid is approximately 3 mol of acid per mole of ruthenium halide.

From a practical viewpoint, the preparation of the complex is performed by mixing, in any order, the ruthenium halide (preferably ruthenium III chloride), the tin halide (preferably tin II chloride) and the strong acid (preferably hydrochloric acid).

The reaction mixture is brought to a temperature ranging from 60° C. to 100° C. and preferably between 70° C. and 95° C.

The duration of this operation may vary widely, and it is pointed out, for illustrative purposes, that a duration ranging from 1 to 3 hours is entirely suitable.

The complex forms quite rapidly, but remains in solution.

Next, if necessary, the temperature is returned to room temperature, i.e. to a temperature usually between 15° C. and 25° C.

The complex solution thus obtained serves to prepare the catalyst of the invention, in particular to deposit the active phase onto the support.

According to a first variant of the process of the invention, the solution of the complex obtained previously is used in the case of preparing a supported catalyst, to deposit the active phase onto the support according to an impregnation technique.

From a practical viewpoint, the metals are deposited onto the support by impregnating said support with the solution of the complex obtained according to the process described above.

The aqueous impregnation solution comprises the ruthenium-tin complex in a proportion of from 1% to 20% by mass of ruthenium.

In practical terms, the impregnation may be performed by spraying onto the support in motion, for example via the rotation of a bezel, the solution comprising the ruthenium-tin complex.

It is also possible to start with a support resulting from an agglomeration of its particles according to well-known techniques, for example extrusion or pelletizing by pressing, and then to impregnate the support by dipping it into the solution of said complex.

According to one preferred variant of the invention, the impregnation is performed “dry”, i.e. the total volume of the solution of complex used is approximately equal to the pore volume presented by the support. The determination of the pore volume may be performed according to any known technique, especially according to the mercury porosimetry method (standard ASTM D 4284-83) or by measuring on a sample the amount of water it absorbs.

In a following step, the impregnated support is then subjected to a reduction operation.

A preferred variant of the invention consists in performing a preliminary drying step.

The drying is usually performed in air at a temperature that may range from room temperature, for example 20° C., up to 100° C.

The duration of the drying is continued until a constant weight is obtained.

Generally, it ranges from 1 to 24 hours, according to the chosen temperature.

In a following step, the reduction of the complex is performed by placing the impregnated support in contact with the reducing agent.

It is possible to envision a chemical reducing agent, but this does not present any specific advantage. Thus, the reduction is preferentially performed with hydrogen.

The hydrogen may be injected at atmospheric pressure or under a slight pressure, for example from 0.5 to 10 bar and preferably between 1 and 2 bar.

The hydrogen may also be diluted with an inert gas such as nitrogen or helium.

Advantageously, the reduction reaction is performed at a temperature of at least 400° C., preferably between 400° C. and 600° C. and even more preferentially between 400° C. and 500° C.

It is understood that the reduction may also be performed during the use of the catalyst in the case where it is used in a reaction for reducing a substrate in the presence of hydrogen.

Thus, the catalyst obtained may be used in the lactone preparation process according to the invention.

When the preparation of the lactone does not take place immediately after the preparation of the catalyst, it may be desirable to perform its activation before use, as described hereinbelow.

According to another variant of the process of the invention, but which is not preferred, the solution of the complex obtained previously may be used to deposit the active phase onto the support via the precipitation technique.

Thus, another mode of preparation, when the support is in powder form, for instance alumina, silica or an abovementioned metal oxide, consists in adding the support to the solution of the complex obtained, performing the hydrolysis of the complex obtained previously and then separating out the solid obtained, preferably by filtration, and blending and extruding it. A catalyst put into form is thus obtained.

The hydrolysis of the complex is obtained by adding water. The amount of water used is not critical: it generally represents from 1 to 100 times the weight of the complex.

Following this hydrolysis, the complex precipitates and is separated out and put into form as described above.

The catalyst thus obtained may be subjected, as described previously for the impregnated support, to a drying and reduction operation and, if need be, may be activated during its use.

The process of the invention is performed in the gas phase.

This term means that the dicarboxylic acid is vaporized under the reaction conditions, but the process does not exclude the presence of a possible liquid phase resulting either from the physical properties of the dicarboxylic acid or from an implementation under pressure or the use of an organic solvent.

Advantageously, the reaction is performed at a temperature of between 270° C. and 450° C. and even more preferentially between 300° C. and 400° C. It is understood that the temperature is adapted by a person skilled in the art as a function of the starting acid, and of the desired reaction rate.

Moreover, it may be particularly advantageous to perform preactivation of the catalyst, by high raising of the temperature. In particular, the catalyst may be subjected beforehand to temperatures close to about 500° C. and preferentially 450° C. The activation is advantageously performed under a stream of hydrogen.

The hydrogen may be injected at atmospheric pressure or under a slight pressure that is compatible with the vapor phase (a few bar, for example from 0.5 to 10 bar). The hydrogen may also be diluted with an inert gas such as nitrogen or helium.

Advantageously, per 1 ml of catalyst, the hydrogen is injected at a flow rate of between 0.1 and 10 liters per hour, and the acid at a liquid flow rate of not more than 10 ml/h and preferably between 0.5 and 5 ml/h.

A practical way of performing the present invention consists in introducing into a reactor a desired amount of catalyst. The temperature of the reactor is then raised under a stream of hydrogen up to a given value, preferably 450° C.-500° C., enabling the catalyst to be activated, and is then returned to the reaction temperature, preferably 300° C.-400° C. The acid is then injected at the desired flow rate and the lactone formed is recovered.

The contact time, which is defined as the ratio between the apparent volume of catalyst and the flow rate of the gas stream (which includes the carrier gas), may vary widely, and is usually between 0.2 and 50 seconds. The contact time is preferably chosen between 0.4 and 10 seconds.

In practice, the reaction is readily performed continuously by passing the gas stream through a tubular reactor containing the catalyst.

The process begins by preparing the catalytic bed, which consists of the catalytic active phase which is deposited onto a support (for example sintered glass or a grate), which allows circulation of the gases without elution of the catalyst. Next, the dicarboxylic acid is placed in contact with the catalyst according to several possible variants.

A first embodiment consists in injecting the acid after it has been vaporized by heating.

Another way of executing the invention is to inject the dicarboxylic acid as a solution in an organic solvent.

Thus, use may be made of an organic solvent which is chosen such that it dissolves the dicarboxylic acid used under the reaction conditions.

Solvents that may be mentioned in particular include polar, protic or aprotic organic solvents.

More particular examples that may especially be mentioned include water, alcohols (for example methanol or ethanol) and ethers (for example dimethoxyethane).

Several solvents may also be used.

The amount of solvent is generally such that the dicarboxylic acid (I) represents from 30% to 60% of the mass of the reaction mixture (acid+solvent).

At the end of the reaction, a gas stream is recovered comprising the lactone, the excess hydrogen, the starting dicarboxylic acid, if any, and an organic solvent.

The lactone is recovered from this gas stream according to the techniques commonly used.

Said stream may be distilled directly at the end of the reaction, and generally produces hydrogen, the optional solvent and then the lactone in the distillation headstock, and the dicarboxylic acid in the distillation tailstock.

When the solvent used is an alcohol, the ester which it forms with the dicarboxylic acid is also obtained. Said ester generally distils off after the alcoholic solvent and before the lactone.

Another variant consists in condensing said stream, for example by cooling with a heat-exchange liquid (for example water at 20° C.), and the lactone is then recovered from the condensed stream by distillation or by liquid-liquid extraction.

Examples of implementation of the invention, which are given for illustrative purposes and with no limiting nature, are given below.

In the examples, the degree of conversion and the yield obtained are defined.

The degree of conversion (DC) corresponds to the ratio between the number of moles of substrate [dicarboxylic acid] converted and the number of moles of substrate [dicarboxylic acid] employed.

The reaction yield (RY) corresponds to the ratio between the number of moles of product formed (lactone) and the number of moles of substrate [dicarboxylic acid] employed.

### EXAMPLES

### Examples of Preparation of Catalysts

To begin with, the preparation of the solutions of ruthenium-tin complexes, which will subsequently be used to prepare the catalysts, is detailed.

Preparation of a Solution of Tin-Ruthenium Complex with an Atomic Ratio Sn/Ru=4

160 ml of 3 N hydrochloric acid are placed in a 250 ml glass reactor and 23.0 g of RuCl₃.xH₂O with x equal to about 2 and having a ruthenium titer of 42% are added.

A solution is obtained by stirring, and 85.6 g of SnCl₂.2H₂O are then added.

The medium is then heated with stirring to 90° C. and these conditions are maintained for 1 hour.

The complex solution is then cooled to room temperature.

Preparation of a Solution of Tin-Ruthenium Complex with an Atomic Ratio Sn/Ru=2

The previous procedure is reproduced, but using, for 23 g of RuCl₃.xH₂O, only 42.8 g of SnCl₂.2H₂O.

### Catalyst 1

Preparation of Catalyst on α-Alumina

40 g of α-alumina beads with a diameter of between 2 and 4 mm are impregnated via the dry impregnation technique with 15 ml of solution of tin-ruthenium complex with Sn/Ru=2.

The beads are then dried in a ventilated oven to constant weight.

10 g of impregnated beads are then placed in a tubular glass reactor 22 mm in diameter.

A stream of 3 l/h of hydrogen is then passed through this bed of catalyst while heating gradually to 450° C.

These conditions are then maintained for at least 5 hours.

The catalyst is then cooled to room temperature and stored in this form.

### Catalyst 2

Preparation of Catalyst on α-Alumina

40 g of α-alumina beads with a diameter of between 2 and 4 mm are impregnated via the dry impregnation technique with 15 ml of solution of tin-ruthenium complex with Sn/Ru=4.

The beads are then dried in a ventilated oven to constant weight.

10 g of impregnated beads are then placed in a tubular glass reactor 22 mm in diameter.

A stream of 3 l/h of hydrogen is then passed through this bed of catalyst while heating gradually to 450° C.

These conditions are then maintained for at least 5 hours.

The catalyst is then cooled to room temperature and stored in this form.

### Catalyst 3

Preparation of Catalyst on Silica

The procedure used for preparing catalyst 1 is repeated, but using a Degussa OX 50 silica preformed in extruded form.

### Catalyst 4

Preparation of Catalyst on Silica

The procedure used for preparing catalyst 2 is repeated, but using a Degussa OX 50 silica preformed in extruded form.

### Catalyst 5

Preparation of Catalyst on Titanium Oxide

The procedure used for preparing catalyst 1 is repeated, but using a commercial pelletized anatase titanium oxide.

### Catalyst 6

Preparation of Catalyst on Titanium Oxide

The procedure used for preparing catalyst 1 is repeated, but using a commercial pelletized anatase titanium oxide.

### Examples of Preparation of Lactones

### Example 1

### Preparation of δ-Valerolactone

10 ml of catalyst 1 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of an aqueous glutaric acid solution at 40% w/w at a flow rate of 6 ml/h is commenced.

The reaction gas stream is then condensed in a receiver immersed in an ice-water bath.

After injection for 10 hours under these conditions, the condensates are analyzed by gas chromatography (GC).

For a degree of conversion of 75%, a 55% yield of δ-valerolactone is obtained.

### Example 2

### Preparation of δ-Valerolactone

10 ml of catalyst 1 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of a methanolic glutaric acid solution at 50% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 100%, a 55% yield of δ-valerolactone is obtained.

### Example 3

### Preparation of δ-Valerolactone

10 ml of catalyst 1 reduced beforehand at 450° C. and 5 ml of glass powder used as static mixer vaporizer are placed in a vertical glass reactor 22 mm in diameter.

The catalytic bed is heated under a stream of 10 l/h of hydrogen to 375° C.

After stabilizing the catalytic bed under these conditions for 30 minutes, injection onto the catalytic bed, by means of a syringe pump, of a methanolic glutaric acid solution at 50% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 100%, a 65% yield of δ-valerolactone is obtained.

### Example 4

### Preparation of δ-Valerolactone

10 ml of catalyst 2 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of an aqueous glutaric acid solution at 40% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 65%, a 32% yield of δ-valerolactone is obtained.

### Example 5

### Preparation of δ-Valerolactone

10 ml of catalyst 2 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of a methanolic glutaric acid solution at 50% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 100%, a 55% yield of δ-valerolactone is obtained.

### Example 6

### Preparation of δ-Valerolactone

10 ml of catalyst 4 reduced beforehand at 450° C. and 5 ml of glass powder used as static mixer vaporizer are placed in a vertical glass reactor 22 mm in diameter.

The catalytic bed is heated under a stream of 10 l/h of hydrogen to 375° C.

After stabilizing the catalytic bed under these conditions for 30 minutes, injection onto the catalytic bed, by means of a syringe pump, of a methanolic glutaric acid solution at 50% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 100%, a 65% yield of δ-valerolactone is obtained.

### Example 7

### Preparation of δ-Valerolactone

10 ml of catalyst 1 reduced beforehand at 450° C. and 5 ml of glass powder used as static mixer vaporizer are placed in a vertical glass reactor 22 mm in diameter.

The catalytic bed is heated under a stream of 101/h of hydrogen to 375° C.

After stabilizing the catalytic bed under these conditions for 30 minutes, injection onto the catalytic bed, by means of a syringe pump, of an aqueous glutaric acid solution at 40% w/w at a flow rate of 10 ml/h is commenced.

The reaction gas stream is then condensed in a receiver immersed in an ice-water bath.

After injection for 5 hours under these conditions, the condensates are analyzed by GC.

For a conversion of 78%, a 42% yield of δ-valerolactone is obtained.

### Example 8

### Preparation of δ-Valerolactone

10 ml of catalyst 1 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of a glutaric acid solution at 30% w/w in dimethoxyethane at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 45%, a 12% yield of δ-valerolactone is obtained.

### Example 9

### Preparation of Caprolactone

10 ml of catalyst 4 reduced beforehand at 450° C. and 5 ml of glass powder used as static mixer vaporizer are placed in a vertical glass reactor 22 mm in diameter.

The catalytic bed is heated under a stream of 5 l/h of hydrogen to 375° C.

After stabilizing the catalytic bed under these conditions for 30 minutes, injection onto the catalytic bed, by means of a syringe pump, of an aqueous adipic acid solution at 2% w/w at a flow rate of 10 ml/h is commenced.

The reaction gas stream is then condensed in a receiver immersed in an ice-water bath.

After injection for 5 hours under these conditions, the condensates are analyzed by GC.

For a degree of conversion of 100%, an 85% yield of caprolactone is obtained.

### Example 10

### Preparation of δ-Valerolactone

Example 4 is repeated, using catalyst 5 prepared on titanium oxide.

For a degree of conversion of 25%, a 12% yield of valerolactone is obtained under these conditions.

### Example 11

### Preparation of δ-Valerolactone

Example 4 is repeated, using catalyst 6 prepared on titanium oxide.

For a degree of conversion of 32%, a 15% yield of valerolactone is obtained under these conditions.

### Example 12

### Preparation of 2-Hydroxy-γ-Butyrolactone

10 ml of catalyst 4 reduced beforehand at 450° C. and 5 ml of glass powder used as static mixer vaporizer are placed in a vertical glass reactor 22 mm in diameter.

The catalytic bed is heated under a stream of 5 l/h of hydrogen to 300° C.

After stabilizing the catalytic bed under these conditions for 30 minutes, injection onto the catalytic bed of an aqueous malic acid solution at 30% w/w at a flow rate of 8 ml/h is commenced.

The reaction gas stream is then condensed in a receiver immersed in an ice-water bath.

After injection for 5 hours under these conditions, the condensate is analyzed by GC and HPLC.

For a degree of conversion of 85%, a 55% yield of 2-hydroxy-γ-butyrolactone is obtained.

### Comparative Example A

### Preparation of δ-Valerolactone

10 ml of catalyst prepared by the same procedure as that used for preparing catalyst 4, but using a solution of tin-ruthenium complex with an atomic ratio Sn/Ru=6, are placed in a vertical glass reactor 22 mm in diameter.

Under these impregnation conditions and after reduction at 450° C., a catalyst containing only the alloy Ru₃Sn₇/SiO₂ is thus obtained.

5 ml of glass powder used as static mixer vaporizer are added over the catalytic bed.

The catalytic bed is heated to 375° C. under a stream of 5 l/h of hydrogen, and, after stabilizing the catalytic bed for 30 minutes, injection of an aqueous glutaric acid solution at 40% w/w at a flow rate of 1 ml/h is commenced.

The reaction gas stream is condensed in a receiver immersed in an ice-water bath.

After injection for 5 hours, the condensate is analyzed by GC.

For a conversion of 21%, an 8% yield of δ-valerolactone is obtained.

1. A process for preparing a lactone, wherein the process comprises reducing a dicarboxylic acid using hydrogen, in a gas phase and with an effective amount of a catalyst present, the catalyst comprising a ruthenium-tin active phase comprised of at least an alloy Ru₂Sn₃ and of an alloy Ru₃Sn₇.

2. The process as described by claim 1, wherein the dicarboxylic acid used corresponds to formula (I) below:
HOOC—R—COOH (I)
wherein in said formula (I), R represents a substituted or unsubstituted divalent group, comprising a linear sequence of atoms in a sufficient number to form the desired lactone.

3. The process as described by claim 1, wherein the dicarboxylic acid used corresponds to formula (I) in which the group R comprises a linear sequence of 2 to 8 atoms.

4. The process as described by claim 1, wherein the dicarboxylic acid used corresponds to formula (I) in which the group R has a total carbon condensation ranging from 2 to 15 carbon atoms, and comprises a linear sequence of 2 to 8 atoms which is then included in a ring obtained.

5. The process as described by claim 1, wherein the dicarboxylic acid used corresponds to formula (I) in which the group R represents:
a saturated or unsaturated, linear or branched aliphatic group,
a saturated or unsaturated, linear or branched aliphatic group in which two vicinal carbon atoms optionally form a ring.

6. The process as described by claim 1, wherein the dicarboxylic acid of formula (I) used is selected from the group consisting of:
succinic acid,
2-ethylsuccinic acid,
malic acid,
glutaric acid,
2-methylglutaric acid,
2-ethylglutaric acid,
adipic acid,
2-methyladipic acid,
3-methyladipic acid,
4-methyladipic acid,
5-methyladipic acid,
2,2-dimethyladipic acid,
3,3-dimethyladipic acid,
2,2,5-trimethyladipic acid,
2,5-dimethyladipic acid,
pimelic (heptanedioic) acid,
2-methylpimelic acid,
2,2-dimethylpimelic acid,
3,3-dimethylpimelic acid,
2,5-dimethylpimelic acid,
2,2,5-trimethylpimelic acid,
azelaic acid,
sebacic acid, and
1,2-phenylenediacetic acid.

7. The process as described by claim 1, wherein the active phase of the catalyst comprises ruthenium and tin in an Sn/Ru atomic ratio at least equal to 3/2 but less than 7/3.

8. The process as described by claim 7, wherein the active phase of the catalyst comprises ruthenium and tin in an Sn/Ru atomic ratio at least equal to 9/5 but less than 2/1.

9. The process as described by claim 1, wherein the active phase is deposited onto a support.

10. The process as described by claim 1, wherein the reduction of the dicarboxylic acid is performed at a temperature of from 270° C. to 450° C.

11. The process as described by claim 1, wherein the hydrogen is injected at atmospheric pressure or under a slight pressure, optionally diluted with an inert gas.

12. The process as described by claim 1, wherein the activation of the catalyst is performed at the start of the reaction by heating to a temperature of from 450° C. to 500° C.

13. A cyclizing hydrogenation catalyst comprising a ruthenium-tin active phase, wherein:
the ruthenium-tin active phase is comprised of an Ru₂Sn₃ alloy and an Ru₃Sn₇ alloy,
wherein the Ru₂Sn₃ alloy phase represents at least 75% by mass of the active phase, and
at least 90% by mass of the ruthenium is in an Ru₂Sn₃ and Ru₃Sn₇ alloy form.

14. The catalyst as described by claim 13, wherein the Ru₂Sn₃ alloy phase represents at least 90% by mass of the two alloy phases Ru₂Sn₃ and Ru₃Sn₇.

15. The catalyst as described by claim 13, wherein during the active phase at least 95% by mass of the ruthenium is in an Ru₂Sn₃ and Ru₃Sn₇ alloy form.

16. The catalyst as described by claim 13, wherein during its active phase, the catalyst comprises ruthenium and tin in an Sn/Ru atomic ratio at least equal to 3/2 and less than 7/3.

17. The catalyst as described by claim 13, wherein during its active phase, the catalyst is deposited onto a support.

18. The catalyst as described by claim 17, wherein the ruthenium content of the supported catalyst is selected from 1% to 8% by mass.

19. A cyclizing hydrogenation catalyst comprising a ruthenium-tin active phase, wherein the cyclizing hydrogenation catalyst is obtained according to a process comprising a step for the preparation of complex(es) corresponding to formula (A) below:
[Ru(SnX₃)₆₋ₙXₙ]⁴⁻ (A)
wherein in said formula (A), X represents a halogen atom, and n is a number equal to 1 or 2, said complex being obtained by reacting a ruthenium halide and a tin halide used in amounts such that the ratio between the number of moles of tin halide and the number of moles of ruthenium halide ranges from 1 to 5, in the presence of an acid, and the reaction mixture is brought to a temperature ranging from 60° C. to 100° C.

20. The cyclizing hydrogenation catalyst as described by claim 19, wherein the active phase is deposited onto a support using the complex obtained according to a precipitation technique or an impregnation technique.

21. The catalyst as described by claim 19, wherein the reduction of the complex is performed by placing the impregnated support in contact with hydrogen, at a temperature of at least 400° C.

22. A method of preparing γ-butyrolactone, δ-valerolactone, caprolactone or 2-hydroxy-γ-butyrolactone, the method comprising preparing the γ-butyrolactone, δ-valerolactone, caprolactone or 2-hydroxy-γ-butyrolactone using the method described by claim 1.

23. The process as described by claim 3, wherein the dicarboxylic acid used corresponds to formula (I) in which the group R comprises a linear sequence of from 2 to 6 atoms.

24. The process as described by claim 3, wherein the dicarboxylic acid used corresponds to formula (I) in which the group R comprises a linear sequence of from 2 to 4 atoms.

25. The process as described by claim 9, wherein the support is a metal oxide.

26. The process as described by claim 25, wherein the metal oxide is selected from the group consisting of an aluminum oxide, a silicon oxide, a titanium oxide, a zirconium oxide and a mixture thereof.

27. The process as described in claim 10, wherein the reduction of the dicarboxylic acid is performed at a temperature of from 300° C. to 400° C.

28. The catalyst as described by claim 16, wherein during its active phase, the catalyst comprises ruthenium and tin in an Sn/Ru atomic ratio at least equal to 9/5 and less than 2/1.

29. The catalyst as described by claim 18, wherein the ruthenium content of the supported catalyst is selected from 2% to 3% by mass.

30. The catalyst as described by claim 17, wherein the support is a metal oxide.

31. The catalyst as described by claim 30, wherein the metal oxide is selected from the group consisting of an aluminum oxide, a silicon oxide, a titanium oxide, a zirconium oxide and a mixture thereof.

32. The cyclizing hydrogenation catalyst as described by claim 19, wherein X is a chlorine atom or a bromine atom.

33. The cyclizing hydrogenation catalyst as described by claim 19, wherein n is equal to 2.

34. The cyclizing hydrogenation catalyst as described by claim 19, wherein the ratio between the number of moles of tin halide and the number of moles of ruthenium halide ranges from 2 to 4.

35. The cyclizing hydrogenation catalyst as described by claim 19, wherein the reaction mixture is brought to a temperature ranging from 70° C. to 95° C.

36. The cyclizing hydrogenation catalyst as described by claim 20, wherein the impregnation technique is dry impregnation.

37. The cyclizing hydrogenation catalyst as described by claim 21, wherein the temperature is from 400° C. to 600° C.

38. The cyclizing hydrogenation catalyst as described by claim 21, wherein the temperature is from 400° C. to 500° C.
